(19) United States
(12) Patent Application Publication
(10) Pub. No.: US 2019/0352704 A1
(43) Pub. Date: Nov. 21, 2019

(54) BENIGN THYROID NODULE-SPECIFIC GENE

(71) Applicant: Guang NING, Shanghai (CN)

(72) Inventors: Guang NING, Shanghai (CN); Weiqing WANG, Shanghai (CN); Lei YE, Shanghai (CN); Xiaoyi ZHOU, Shanghai (CN)

(21) Appl. No.: 16/467,573

(22) PCT Filed: Dec. 7, 2017
(86) PCT No.: PCT/CN2017/114889

§ 371 Date: Jun. 7, 2019

(30) Foreign Application Priority Data
Dec. 7, 2016 (CN) 201611115776.3

(51) Int. Cl. C12Q 1/6827; C12Q 1/6883; C12Q 1/6837; G16H 50/30

(52) U.S. Cl. CPC C12Q 1/6827; C12Q 1/6883; C12Q 2600/156; G16H 50/30; C12Q 1/6837

(57) ABSTRACT

Disclosed are three benign thyroid nodule-specific genes SPOP, EZH1 and ZNF148 and the use thereof in the detection of benign thyroid nodules. Also provided are a method for detecting benign thyroid nodules and a corresponding detection kit.

### TECHNICAL FIELD

This invention belongs to the field of medical testing, and particularly relates to three benign thyroid nodule specific genes.

### TECHNICAL BACKGROUND

With the popularity of conventional thyroid ultrasound, the detection rate of thyroid nodules has increased significantly. A large-scale population survey found that the prevalence of thyroid nodules is highest in women and the elderly, reaching 19-68%. Most new nodules are benign nodules, with less than 5% of nodules diagnosed as malignant. Although high-resolution ultrasound combined with fine-needle aspiration cytology results in an accuracy rate of diagnosis of 85% of benign and malignant thyroid nodules, patients and clinicians are always anxious about whether benign nodules have malignant potential. Therefore, in 2009, the American Thyroid Association (ATA) recommended regular follow-up of benign nodules every 12-18 months, resulting in huge medical resources and social psychosocial burden.

For several years, the molecular mechanism on thyroid nodules is mainly focused on malignant nodules and the molecular mechanism on thyroid cancer. In 2014, The Cancer Genome Atlas (TCGA) Thyroid Cancer Research Group described the genomic characteristics of papillary carcinoma in detail and found that 96.5% of papillary thyroid carcinomas have clear driving gene variants. However, the genetic characteristics of benign thyroid nodules have rarely been reported. There is an urgent need in the field to study the genetic characteristics of benign thyroid nodules.

### SUMMARY OF THE INVENTION

The object of the invention is to provide specific genes for benign thyroid nodules.

In a first aspect of the invention, it provides a kit for detecting a benign thyroid nodule, the kit comprises one or more pairs of primers selected from the group consisting of:

(i) a primer for specifically amplifying a SPOP gene or a transcript, the primer amplifies an amplification product having a length of 80 to 2000 bp and containing the 281th position of SEQ ID NO.: 1;

(ii) a primer for specifically amplifying an EZH1 gene or a transcript, the primer amplifies an amplification product having a length of 80 to 2000 bp and containing the 1712th position of SEQ ID NO.: 3;

(iii) a primer for specifically amplifying a ZNF148 gene or a transcript, the primer amplifies an amplification product having a length of 1000 to 3000 bp and containing positions 1273 to 2871 of SEQ ID NO.: 5.

In another preferred embodiment, the nucleotide sequence of the primer for specifically amplifying the SPOP gene or transcript is as shown in SEQ ID NO.: 7 and 8.

In another preferred embodiment, the nucleotide sequence of the primer for specifically amplifying the EZH 1 gene or transcript is as shown in SEQ ID NO.: 9 and 10.

In another preferred embodiment, the primer that specifically amplifies the ZNF148 gene or transcript is selected from the group consisting of:

(i) The nucleotide sequences of the primer pairs are shown in SEQ ID NO.: 11 and 12;

(ii) The nucleotide sequences of the primer pairs are shown in SEQ ID NO.: 13 and 14;

(iii) The nucleotide sequences of the primer pairs are shown in SEQ ID NO.: 15 and 16;

In another preferred embodiment, the kit further comprises a reagent selected from the group consisting of:

(a) a probe or chip that binds to the C→G mutation at position 281 in SEQ ID NO.: 1;

(b) a restriction endonuclease that recognizes C→G mutation at position 281 in SEQ ID NO.: 1;

(c) a probe or chip that binds to the A→G mutation at position 1712 in SEQ ID NO.: 3;

(d) a restriction endonuclease that recognizes A→G mutation at position 1712 in SEQ ID NO.: 3.

In another preferred embodiment, the mutation includes a single-stranded mutation and a double-stranded mutation.

In another preferred embodiment, the kit further comprises a reagent selected from the group consisting of:

(I) a specific antibody for detecting the P→R mutation at position 94 in SEQ ID NO.: 2;

(II) a specific antibody for detecting the Q→R mutation at position 571 in SEQ ID NO.: 4.

In another preferred embodiment, the kit is used for the auxiliary judgment of benign thyroid nodules.

In another preferred embodiment, the kit is used for the detection of a thyroid nodule tissue sample and/or a blood sample.

In another preferred embodiment, the detection is pre-detection.

In another preferred embodiment, the blood sample comprises a serum and a plasma.

In another preferred embodiment, the detection is performed on Asian population.

In another preferred embodiment, the detection is performed on Chinese population.

In another preferred embodiment, the detection is for determining whether the thyroid nodule is benign.

In another preferred embodiment, the test is for determining that the thyroid nodule is not a malignant thyroid nodule, and preferably for determining that the thyroid nodule is not papillary thyroid cancer.

In a second aspect of the invention, it provides a use of a polynucleotide molecule for the preparation of a kit for detecting benign thyroid nodules; wherein, said polynucleotide molecule comprises:

(i) a SPOP gene, a primer that specifically amplifies a SPOP gene or a transcript, a probe or a chip that specifically binds to a nucleotide sequence of the SPOP gene, that is, the C→G mutation at position 281 in SEQ ID NO.: 1, and/or a specific antibody for detecting the P→R mutation at position 94 in SEQ ID NO.: 2;

(ii) the EZH1 gene, a primer that specifically amplifies the EZH1 gene or transcript, a probe or chip that specifically binds to the nucleotide sequence of the EZH1 gene, ie, the A→G mutation at position 1712 in SEQ ID NO.: 3, and/or a specific antibody for detecting the Q→R mutation at position 571 in SEQ ID NO.: 4; and/or

(iii) a primer that specifically amplifies the ZNF148 gene or transcript, A probe that specifically binds to the nucleotide sequence of the ZNF148 gene, i.e., position 1273-2871 of SEQ ID NO.: 5.

In another preferred embodiment, the kit is used for the auxiliary judgment of benign thyroid nodules.

In another preferred embodiment, the kit further includes a specification in which the following is described:

When the test subject has one or more of the mutations, the thyroid nodules of the test subject are suggested to be benign.

In a third aspect of the invention, it provides a use of a benign thyroid nodule related gene for preparing a reagent or a kit for detecting a benign thyroid nodule, and the benign thyroid nodule related gene comprises the SPOP gene, EZH1 gene, and/or ZNF148 gene.

In another preferred embodiment, the reagent or kit is used to detect the following single nucleotide mutations:

The nucleotide sequence of the SPOP gene: the C→G at poison 281 in SEQ ID NO.: 1.

In another preferred embodiment, the reagent comprises a primer that specifically amplifies a SPOP gene or a transcript, an amplification product containing the mutation site, a probe that specifically binds to the mutation site, and a nucleic acid chip that specifically detects the mutation site.

In another preferred embodiment, the kit comprises instructions for use and one or more of the following reagents:

a container (a) and a primer located within the container that specifically amplifies a SPOP gene or transcript;

a container (b) and a probe located within the container that specifically binds to the mutation site;

a container (c) and a nucleic acid chip within the container that specifically detects the mutation site.

In another preferred embodiment, the SPOP gene is used as a standard or control.

In another preferred embodiment, the reagent or kit is used to detect the following single nucleotide mutations:

The nucleotide sequence of the EZH1 gene: that is, the A→G at position 1712 in SEQ ID NO.: 3.

In another preferred embodiment, the reagent comprises a primer that specifically amplifies an EZH1 gene or a transcript, an amplification product containing the mutation site, a probe that specifically binds to the mutation site, and a nucleic acid chip that specifically detects the mutation site.

In another preferred embodiment, the kit comprises instructions for use and one or more of the following reagents:

a container (a) and a primer in the container that specifically amplifies the EZH1 gene or transcript;

a container (b) and a probe located within the container that specifically binds to the mutation site;

a container (c) and a nucleic acid chip located within the container that specifically detects the mutation site.

In another preferred embodiment, the EZH1 gene is used as a standard or control.

In another preferred embodiment, the reagent or kit is used to detect the following mutations:

The nucleotide sequence of the ZNF148 gene: the mutation at position 1273-2871 in SEQ ID NO.: 5.

In another preferred embodiment, the ZNF148 gene is used as a standard or control.

In a fourth aspect of the invention, it provides a method for non-diagnostic detection of benign thyroid nodule related genes mutation in a sample in vitro, comprising the steps of:

(a) amplifying a sample of the SPOP gene, the EZH1 gene, and/or the ZNF148 gene with a specific primer to obtain an amplification product;

(b) detecting the presence or absence of the following mutation sites in the amplified product:

the nucleotide sequence of the SPOP gene: the C→G at poison 281 in SEQ ID NO.: 1;

the nucleotide sequence of the EZH1 gene: the A→G at poison 1712 in SEQ ID NO.: 3;

the nucleotide sequence of the ZNF148 gene: the mutation at position 1273-2871 in SEQ ID NO.: 5.

In another preferred embodiment, the amplification product is 80-2000 bp in length and comprises position 281 in SEQ ID NO: 1, position 1712 in SEQ ID NO.: 3, and/or the 1273-2871 position in SEQ ID NO.: 5.

In another preferred embodiment, the amplified sample is a thyroid nodule tissue sample.

In a fifth aspect of the invention, it provides a method of detecting a benign thyroid nodule in a subject, the method comprises the steps of:

Detecting the following genes, transcripts and/or proteins in the subject:

SPOP gene, transcript and/or protein, and compared to normal SPOP genes, transcripts and/or proteins,

EZH1 gene, transcript and/or protein, and compared to the normal EZH1 gene, transcript and/or protein,

ZNF148 gene, transcript and/or protein, and compared to the normal ZNF148 gene, transcript and/or protein,

wherein, the difference indicates that the thyroid nodules in the subject are benign.

In another preferred embodiment, detecting genes, transcripts, and/or proteins in a nodule sample of the subject to be tested and compared to the genes, transcripts, and/or proteins in the blood sample of the subject.

In another preferred embodiment, the difference is that the following mutations:

The nucleotide sequence of the SPOP gene is the C→G at position 281 in SEQ ID NO.: 1;

The nucleotide sequence of the EZH1 gene is the A→G at position 1712 in SEQ ID NO.: 3;

The nucleotide sequence of the ZNF148 gene is mutated at positions 1273 to 2871 in SEQ ID NO.: 5.

In another preferred embodiment, the thyroid nodule tissue sample of the subject is tested to detect whether the thyroid nodule of the subject is benign.

It should be understood that in the present invention, any of the technical features specifically described above and below (such as in the Examples) can be combined with each other, thereby constituting new or preferred technical solutions that are not described one by one in the specification.

### DETAILED DESCRIPTION OF THE INVENTION

The inventors have extensively and intensively studied, and for the first time, unexpectedly discovered genes associated with three sexual nodules, namely SPOP gene, EZH1 gene and ZNF148 gene. The experiment shows that SPOP, EZH1 and ZNF148 are mutually dissociated gene mutations that occur in 29.2% of benign nodules, and do not occur in paired PTC (papillary thyroid carcinoma) tumor tissues. The above three benign nodule-related genes provide “excluded” information for malignant thyroid nodules and have an important diagnostic significance in gene mutation detection.

SPOP Gene

The protein encoded by the SPOP gene (NM_001007226) regulates the transcriptional inhibitory activity of death-related protein (DAXX), which interacts with histone deacetylase, core histone, and other histone-associated proteins. In mice, the SPOP-encoded protein binds to the leucine zipper domain of macroH2A1.2, which is an isoform of the H2A histone, enriched on the inactive X chromosome. The BTB/POZ domain of this protein interacts with other proteins, regulates transcriptional repression activity, and interacts with components of the co-inhibition complex of histone deacetylase. Selective splicing of the SPOP gene produces many transcript variants and encodes the same protein.

EZH1 Gene

The protein encoded by the EZH 1 gene (NM_001991) is a part of a non-canonical polycombine inhibitor complex 2 (PRC-2) that regulates the methylation of lysine at position 27 of histone H3 (H3K27), and plays an important role in maintaining the pluripotency and plasticity of embryonic stem cells.

ZNF148 Gene

The protein encoded by the ZNF148 gene (NM_021964) (zinc finger protein 148) belongs to a class of Kruppel-like transcription factors, which both have transcriptional activation and transcription inhibition on its target protein. The low expression of ZNF148 is associated with poor prognosis in colorectal cancer, and the expression of ZNF148 overexpressing clones is significantly reduced in hepatocellular carcinoma cell lines.

Thyroid Nodules

Thyroid nodules are masses in the thyroid gland that move up and down with the thyroid gland as they swallow. They are common clinical conditions and can be caused by a variety of causes. There are many thyroid diseases in the clinic, such as thyroid degeneration, inflammation, autoimmunity and new organisms, which can be expressed as nodules. Thyroid nodules can be single or multiple, and multiple nodules have a higher incidence than single nodules, but the incidence of single nodular thyroid cancer is higher.

Thyroid nodules are classified into benign thyroid nodules and malignant thyroid nodules. Most new nodules are benign nodules, with less than 5% of nodules diagnosed as malignant.

Detection Method, Detection Reagent and Kit

The present invention provides a method for detecting a benign thyroid nodule in a subject by detecting a SPOP gene, an EZH 1 gene, and a ZNF148 gene in a thyroid nodule, and comparing it with a corresponding gene in the blood sample to predict in advance whether the thyroid nodule is benign. The method of the invention can be used to auxiliary diagnostic typing, especially early auxiliary diagnosis.

Specifically, the methods, reagents, and kits of the invention detect the following mutations:

| | | | The |
|---|---|---|---|
| | | | mutant |
| | | The mutant site of | form of |
| | The mutation of amino acid | nucleotide | nucleotide |
| SPOP | P94R(The mutation of 94th | 281th of the gene | C→G |
| gene | P is R, which indicates that | | |
| | the thyroid nodule is benign | | |
| | when it is R) | | |
| EZH1 | Q571R(The mutation of | 1712th of the gene | A→G |
| gene | 571th Q is R, which | | |
| | indicates that the thyroid | | |
| | nodule is benign when it is | | |
| | R) | | |
| ZNF148 | The last exon is nonsense | C1624T and | |
| gene | mutation or frameshift | others; Amino | |
| | mutation | acid mutations: | |
| | | Multiple variations | |
| | | such as Q542X | |
| | | that cause the | |
| | | last exon to | |
| | | be frameshifted or | |
| | | terminated | |

Those skilled in the art know that a large number of analytical techniques are available for detecting the presence or absence of a mutation at the site in the gene. These techniques include, but are not limited to, DNA sequencing, hybridization sequencing; enzymatic mismatch cleavage, heteroduplex analysis, dot hybridization, oligonucleotide arrays (chips), pyrosequencing, Taqman probe detection techniques, molecular beacons, etc.

The test sample used in the present invention is not particularly limited, and for detecting a mutation site, it may be DNA or mRNA extracted from a sample such as a cell or a tissue. Since the mutation of the present invention is mainly present in thyroid nodule cells, it is usually not present in peripheral blood cells. Therefore, the preferred test sample is thyroid nodule cells, and peripheral blood cells can be used as a control.

A part or all of the gene sequence detection of the present invention can be immobilized as a probe on a microarray or a DNA chip (also referred to as a “gene chip” or a “nucleic acid chip”) for analyzing sequence and differential expression analysis of genes in tissues, and gene diagnosis. The corresponding transcripts can also be detected by RNA-polymerase chain reaction (RT-PCR) in vitro amplification using specific primers for the SPOP gene, EZH1 gene, and ZNF148 gene.

Detection can be directed to cDNA as well as to genomic DNA. Mutations of the SPOP gene, EZH1 gene, and ZNF148 gene include point mutations, translocations, deletions, recombinations, and any other abnormalities compared to normal wild-type DNA sequences. Mutations can be detected using established techniques such as Southern blotting, DNA sequence analysis, PCR and in situ hybridization. In addition, mutations may affect the expression of related proteins, so the presence or absence of mutations can be indirectly determined by Northern blotting and Western blotting.

The most convenient method for detecting the mutation site of the present invention is to obtain an amplification product by separately amplifying the SPOP gene, the EZH1 gene, and the ZNF148 gene in the sample by using specific primers of the SPOP gene, the EZH1 gene, and the ZNF148 gene; and then detecting whether the single nucleotide mutation (SNV) of the present invention exists in the amplified product.

Specifically, representative primer sequences that can be used for detection are as follows:

SPOP primer sequence:
(SEQ ID NO.: 7)
F: CCAGATCAAAGCCACAAC
(SEQ ID NO.: 8)
R: CTGGACGATAGAGTAAGACC
EZH1 Primer Sequence:
(SEQ ID NO.: 9)
F: ACACCTGCTTTTTTGACTCG
(SEQ ID NO.: 10)
R: AACCAGTGGAAAGAGAATGC
ZNF148 Last exon Primer sequence:
(SEQ ID NO.: 11)
1) F: TCTTGGTTGACCAAAACCAC
(SEQ ID NO.: 12)
R: GGCCCCTCCTGCAAATTATC
(SEQ ID NO.: 13)
2) F: TTTGGGAGGGTCTGGTTATC
(SEQ ID NO.: 14)
R: CCACATATGAAGAGAGCAAAG
(SEQ ID NO.: 15)
3) F: CAGGCTTTGGACAGAACTAG
(SEQ ID NO.: 16)
R: TACACAGAGTAACCCCACTC

It should be understood that after the present invention first reveals the correlation between the mutation sites of the SPOP gene, the EZH1 gene, and the ZNF148 gene and the benign thyroid nodules, those skilled in the art can conveniently design an amplification product that specifically amplifies the position containing the mutation site, and then determine whether the mutation of the present invention exists by sequencing or the like. Typically, the primers are 15 to 50 bp in length, preferably 20 to 30 bp. Although it is preferred that the primer is fully complementary to the template sequence, those skilled in the art will recognize that in the case of a certain non-complement of the primer and the template (especially the 5′ end of the primer), it is also capable of specific amplification (ie only amplify the desired fragment). Kits containing these primers and methods of using the same are within the scope of the present invention as long as the amplification product amplified by the primer contains the corresponding position of the mutation site of the gene of the present invention.

Although the length of the amplification product is not particularly limited, the length of the amplification product is usually 100 to 2000 bp, preferably 150 to 1500 bp, more preferably 200 to 1000 bp. These amplification products should all contain a single nucleotide mutation (SNV) site of the invention.

The main advantages of the invention include:

(a) The discovery of three benign nodule-related genes, SPOP, EZH1 and ZNF148, provides “excluded” information on papillary thyroid carcinoma;

(b) It is strongly confirmed that most benign nodules are not precancerous and have nothing to do with the occurrence of papillary carcinoma;

(c) In the presence of SPOP, ZNF148, or EZH1 mutations, routine monitoring of benign thyroid nodules may be unnecessary, saving significant medical resources.

The present invention will be further illustrated below with references to the specific examples. It should be understood that these examples are only to illustrate the invention but not to limit the scope of the invention. The experimental methods with no specific conditions described in the following examples are generally performed under the conventional conditions or according to the conditions recommended by the manufacturer. Unless indicated otherwise, parts and percentage are calculated by weight.

General Materials and Methods

Sample Preparation and DNA Extraction

Approved by the Ethics Committee of Ruijin Hospital affiliated to Shanghai Jiao Tong University School of Medicine, and after informed consent, 127 tissue samples were obtained from surgical specimens of 28 patients, including 21 patients with cancerous nodules (both with benign thyroid nodules and papillary thyroid carcinoma) and 8 patients with simple benign nodules. A simple benign nodule is defined as having at least one thyroid nodule and is present for more than 2 years without malignant histological signs. All patients were not treated (radiotherapy or chemotherapy) prior to specimen collection. Patient blood samples were used as germ cell line controls (to identify somatic variations). All tissues were quickly stored in liquid nitrogen for collection and analyzed independently to minimize contamination and interference. After examination of HE-stained sections by an experienced pathologist, DNA was extracted from the pathologically confirmed area (cell density of thyroid papillary carcinoma tissue>80%). Pathological sections were scanned using a digital pathology scanner nanozoomer 2.0-RS (Hamamatsu) and tissue DNA was extracted using the QIAGEN DNeasy Blood & Tissue Kit. For patients with cancerous nodules, benign nodules, papillary carcinoma and normal tissues were collected at the same time; For patients with simple nodules, benign nodules and matched normal thyroid tissue were collected.

Whole Exome Sequencing

A total of 127 tissues DNA from 28 patients were randomly broken into small fragments by ultrasonic tissue homogenizer to construct a sequencing library with an average insert size of 300 bp. Whole exome capture was performed using a SureSelect Human All Exon 50 Mb kit (Agilent Technologies, Santa Clara, Calif.) and further sequenced using an Illumina HiSeq 2500 sequencing system to generate a 100 bp paired sequence. All sample sequencing data was rigorously filtered to obtain high-quality raw sequencing data with an average data volume of 13.26 GB (the average effective coverage of whole exon sequencing was 161×, with a minimum of 130× coverage and a maximum of 180×).

Comment and Naming of Variant Sites

The obtained paired sequences of whole exon sequencing were sequence aligned with the human reference genome (hg19) using BWA software (version 0.7) using its default parameter settings. Repetitive products resulting from PCR amplification were removed using the Picard tool (version 1.1). In a localized region with an insertion or deletion mutation, the sequence alignment is repeated and the base quality score is corrected. After these analyses, the BAM file (binary alignment file) was finally obtained, and the mutation site was identified using the UnifiedGenotyper module in the GATK software package. In order to compare the mutations of specific patient-matched tissues, a single normal tissue-multiple tumor sample strategy was used based on the GATK combined recognition of somatic mutation sites. To avoid errors in sequencing or alignment, the following criteria were used: 1) both tissue and control blood samples must have complete, sufficient sequence coverage (at least 10× depth); 2) at least 10% of the sequences covering a site in the tissue support mutated bases (if the local depth is >50 times, set to 5%); 3) in the tissue, the mutations were found to be at least 3 times in the sequencing data. 4) For each possible somatic mutation site, the chi-square test was used to detect the allelic depth and frequency of multiple tissues and control blood samples; 5) exclude sites that also show mutations in control blood samples (more than 2 sequences supported mutations in the blood samples). In the subsequent analysis, common mutations in the dbSNP database (build 142), thousands of human genomes (minimum allele frequency MAF>5%), exome aggregation consortium database (MAF>1%), mutations in intron regions and intergenic regions were excluded. Single-base mutations (SNVs) and insertion-deletion mutations of somatic mutations were annotated using ANNOVAR software to identify the genes located and the proteins that may be affected.

Mutation Analysis

Assuming that the protein coding gene of the human exon is 30 Mb in total and completely covered, the mutation density is calculated. The somatically mutated base uses the aforementioned SNV analysis results. When analyzing common mutations in benign tumors, the mutant sequences of matched tumors and benign nodules were compared to find important mutations unique to benign tumors.

Verification of Mutations Using PCR and Sanger Generation Sequencing

The mutation sites found in the whole exome sequencing were further verified by PCR using a 96-well plate (GeneAmp PCR System 9700, supplied by Biosystems, France), and 20 ng of DNA template was used for each reaction. The PCR product was sequenced by a 3730×1 DNA Analyzer (Applied Biosystems, Courtaboeuf, France) and analyzed using sequencing analysis software (Applied Biosystems, version 5.2, Courtaboeuf, France). All positive mutations were confirmed by an artificial check based on the original sequenced trace file.

Expand the Population to Assess the Frequency of Important Mutations in Benign Nodules

A total of 328 cases of benign thyroid nodular tissue of 259 patients with liquid nitrogen were collected. The genomic DNA was extracted as described above, and the SPOPP94R and EZH1Q571R hot spot mutation sites were designed, and the exon fragment of the primer pair site was designed for PCR amplification, and the PCR product was sequenced by Sanger sequencing method. The variation frequency was calculated by artificially checking the variation based on the original sequenced trace file. For the ZNF148 gene (NM_021964), since the whole exome found multiple mutations in the last exon, the PCR product was designed and the flanking of all coding regions and intron-exon junction regions was sequenced one by one, and the variation of the entire ZNF148 coding region was counted.

### EXAMPLE 1

### Mutation Analysis of Thyroid Nodules

Whole exon sequencing and mutation analysis were performed on 127 tissue samples from 28 patients collected by surgery. Samples from 21 patients with cancerous nodules (both thyroid benign nodules and thyroid papillary PTC) were PTC group samples, and samples from 8 patients with simple benign nodules were non-PTC group samples.

A total of 734 individual cell mutations of 535 genes were found in a pool of 28 patients. The frequency of mutations in benign nodules (0.36 mutations per Mb) was actually higher than papillary carcinoma (0.33 mutations per Mb) (P=0.58). By comparison, there was no significant difference in the frequency of mutations between benign nodules from the PTC group and the non-PTC group (0.34 per Mb and 0.38 mutations per Mb, P=0.70, t-test).

Among the benign nodules of 28 patients, the most common frequent mutations were SPOP (detected in 4 patients, 14.3%), EZH1 (detected in 3 patients, 10.7%) and ZNF148 (detected in 6 patients, 21.4%). Both SPOP and EZH1 are hotspot mutations, which are (P94R) and (Q571R), respectively; the mutation of ZNF148 is located in the last exon and is a nonsense mutation or a frameshift mutation.

### EXAMPLE 2

### Expanded Analysis of Benign Thyroid Nodule Specific Genes

To expand the sample to verify the specific relationship between these three genes and benign thyroid nodules, 231 patients with additional benign thyroid nodules were tested. The results showed that 29 of the 231 patients (11.2%) had SPOPP94R mutations, 24 had EZH1Q571R mutations, and 14 had ZNF148 mutations (5.4%), each of which did not intersect.

Analysis of the information in the TCGA database of thyroid cancer showed that the incidence of the above three genes was extremely low (only one SPOP, two EZH1, two ZNF148 were found in several thousand samples), and both were accompanied by known PTC-driven mutations.

Discussion

Thyroid nodule formation is a primary early stimulator of goiter. Causes of nodule formation include iodine deficiency, nutritional goiter or autoimmune diseases. In contrast, thyroid nodules resulting from local proliferation of follicular epithelial cells form monoclonal proliferation and are caused by somatic mutations. In a normal thyroid nodule, only a small fraction of TSHR, GNAS, or RAS family genes have somatic mutations. In addition, it is unclear whether there are specific subpopulations of precancerous lesions in multinodular disease. Gene mutations in benign thyroid nodules were first described using whole exome sequencing. Interestingly, the inventors found that although the frequency of gene mutations in benign nodules and papillary carcinomas is similar, the specific genes are different. SPOP, EZH1, and ZNF148 are mutually dissociated gene mutations that occur only in 29.2% of benign nodules and do not occur in paired PTC tumor tissues. The expanded sample was validated in 259 benign nodules, and 25.8% of the nodules contained these three gene mutations. Although these three genes are involved in tumor-associated cell biological behavior, the inventors performed functional experiments in thyroid cell lines, and found that these three genes only promote proliferation, but do not affect the invasion function. The above findings suggest that these three gene mutations are involved in the formation of benign thyroid nodules, but do not lead to their transformation into tumors. At present, the gene mutation detection of thyroid nodules contains only thyroid cancer conversion-related genes for “inclusion” detection; the inventors discovered three benign nodule-related genes, SPOP, EZH1 and ZNF148, which provide “excluded” information and have important diagnostic significance in gene mutation detection.

All literatures mentioned in the present application are incorporated by reference herein, as though individually incorporated by reference. Additionally, it should be understood that after reading the above teaching, many variations and modifications may be made by the skilled in the art, and these equivalents also fall within the scope as defined by the appended claims.

1. A kit for detecting a benign thyroid nodule, the kit comprises one or more pairs of primers selected from the group consisting of:
(i) a primer for specifically amplifying a SPOP gene or a transcript, the primer amplifies an amplification product having a length of 80 to 2000 bp and containing the 281th position of SEQ ID NO.: 1;
(ii) a primer for specifically amplifying an EZH1 gene or a transcript, the primer amplifies an amplification product having a length of 80 to 2000 bp and containing the 1712th position of SEQ ID NO.: 3;
(iii) a primer for specifically amplifying a ZNF148 gene or a transcript, the primer amplifies an amplification product having a length of 1000 to 3000 bp and containing positions 1273 to 2871 of SEQ ID NO.: 5.

2. The kit of claim 1, wherein the kit further comprises a reagent selected from the group consisting of:
(a) a probe or chip that binds to the C→G mutation at position 281 in SEQ ID NO.: 1;
(b) a restriction endonuclease that recognizes C→G mutation at position 281 in SEQ ID NO.: 1;
(c) a probe or chip that binds to the A→G mutation at position 1712 in SEQ ID NO.: 3;
(d) a restriction endonuclease that recognizes A→G mutation at position 1712 in SEQ ID NO.: 3.

3. The kit of claim 1, wherein the kit further comprises a reagent selected from the group consisting of:
(I) a specific antibody for detecting the P→R mutation at position 94 in SEQ ID NO.: 2;
(II) a specific antibody for detecting the Q→R mutation at position 571 in SEQ ID NO.: 4.

4. The kit of claim 1, wherein the kit is used for the auxiliary judgment of benign thyroid nodules.

5. The kit of claim 1, wherein the kit further includes a specification in which the following is described:
When the test subject has one or more of the mutations, the thyroid nodules of the test subject are suggested to be benign.

6-9. (canceled)

10. A method for detection of benign thyroid nodule related genes mutation in vitro in a sample, comprising the steps of:
(a) amplifying a sample of the SPOP gene, the EZH1 gene, and/or the ZNF148 gene with a specific primer to obtain an amplification product;
(b) detecting the presence or absence of the following mutation sites in the amplified product:
the nucleotide sequence of the SPOP gene: the C→G at poison 281 in SEQ ID NO.: 1;
the nucleotide sequence of the EZH1 gene: the A→G at poison 1712 in SEQ ID NO.: 3;
the nucleotide sequence of the ZNF148 gene: the mutation at position 1273-2871 in SEQ ID NO.: 5.

11. A method of detecting a benign thyroid nodule in a subject, the method comprises the steps of:
Detecting the following genes, transcripts and/or proteins in the subject:
SPOP gene, transcript and/or protein, and compared to normal SPOP genes, transcripts and/or proteins,
EZH1 gene, transcript and/or protein, and compared to the normal EZH1 gene, transcript and/or protein,
ZNF148 gene, transcript and/or protein, and compared to the normal ZNF148 gene, transcript and/or protein,
wherein, the difference indicates that the thyroid nodules in the subject are benign.

12. The method of claim 11, wherein detecting genes, transcripts, and/or proteins in a nodule sample of the subject to be tested and compared to the genes, transcripts, and/or proteins in the blood sample of the subject.

13. The method of claim 11, wherein the difference is that the following mutations:
The nucleotide sequence of the SPOP gene is the C→G at position 281 in SEQ ID NO.: 1;
The nucleotide sequence of the EZH1 gene is the A→G at position 1712 in SEQ ID NO.: 3;
The nucleotide sequence of the ZNF148 gene is mutated at positions 1273 to 2871 in SEQ ID NO.: 5.

14. The method of claim 11, wherein the thyroid nodule tissue sample of the subject is tested to detect whether the thyroid nodule of the subject is benign.


SEQUENCE LISTING (16 sequences)

SEQ ID NO: 1 - DNA, 3585 bp, Homo sapiens
gaggaggccgcgcggggtggggtctggcggtacgcgctggctgcgtcgacgtgctgacgccatgacgccccggctggtgtgtgtcggtgtgtatgtgtgtgtgtgagtgtgcgcgctccgagtgtgtgtgtatttgtgtatcggcggtcccgcaggtcccggatgttgcggacagtatgaggcaagcgcagggggacggggaccagcagctgtcgccgccgctctcaggtgagtggggggaggagagtcgaggtttcttttttccttttttttttgagatcgagtcttgctctgtcacccaggctggagtgcagtggcgcgatctcagctcactgccacctttgcctcctgggttcaagcgattcttctgcctcagcctcccgagtagctgggattacaggtgagtgccaccatgcctggctaattttcttgcttcttggatctgaccagggtgaagagggaacagaaatctttgccccctgactttggaaatctcgtttaaccttcaaactggcgatgtcaagggttccaagtcctccacctccggcagaaatgtcgagtggccccgtagctgagagttggtgctacacacaggtaagttgaagttttcagcctgtgattgcttcctgtttttctatcaacagatcaaggtagtgaaattctcctacatgtggaccatcaataactttagcttttgccgggaggaaatgggtgaagtcattaaaagttctacattttcatcaggagcaaatgataaactgaaatggtgaggaagaatacgtctaactgtattttttttctatctgttttggacaggtgtttgcgagtaaaccccaaagggttagatgaagaaagcaaagattacctgtcactttacctgttactggtcagctgtccaaagagtgaagttcgggcaaaattcaaattctccatcctgaatgccaagggagaagaaaccaaagctatgggtaaatgttctcctctttgttcaacatgactttttttttccccaccccagagagtcaacgggcatataggtttgtgcaaggcaaagactggggattcaagaaattcatccgtagagattttcttttggatgaggccaacgggcttctccctgatgacaagcttaccctcttctgcgaggtgagtccttgtattctgctgagacgcttgtgtttccttgtctttcacaggtgagtgttgtgcaagattctgtcaacatttctggccagaataccatgaacatggtaaaggttcctgagtgccggctggcagatgagttaggaggactgtgggagaattcccggttcacagactgctgcttgtgtgttgccggccaggaattccaggctcacaaggctatcttagcaggttggtatttattcatgaggaattttgcttgtttctctttgactttgtagctcgttctccggtttttagtgccatgtttgaacatgaaatggaggagagcaaaaaggtatgtaacaagatgaagacatgtcctcatattcagtttttctggcatagaatcgagttgaaatcaatgatgtggagcctgaagtttttaaggaaatgatgtgcttcatttacacggggaaggctccaaacctcgacaaaatggctgatgatttgctggcagctgctgacaaggtaagataagaatagaaaaataatctgacagcagtgcttgtgttttacagtatgccctggagcgcttaaaggtcatgtgtgaggatgccctctgcagtaacctgtccgtggagaacgctgcagaaattctcatcctggccgacctccacagtgcagatcagttgaaaactcaggcagtggatttcatcaactagtgagttggcatcttcaaagttcttacccatttctccacatttctcctagtcatgcttcggatgtcttggagacctctgggtggaagtcaatggtggtgtcacatccccacttggtggctgaggcataccgctctctggcttcagcacagtgcccttttctgggacccccacgcaaacgcctgaagcaatcctaagatcctgcttgttgtaagactccgtttaatttccagaagcagcagccactgttgctgccactgaccaccaggtagacagcgcaatctgtggagcttttactctgttgtgaggggaagagactgcattgtggccccagacttttaaaacagcactaaataacttgggggaaacggggggagggaaaatgaaatgaaaaccctgttgctgcgtcactgtgttccctttggcctggctgagtttgatactgtggggattcagtttaggcgctggcccgaggatatcccagcggtggtacttcggagacacctgtctgcatctgactgagcagaacaaatcgtcaggtgcctggagcaaaaaggaaaaaaaaaaaagaaaggacattgagttttaacagaagggaaaaggaaagaagaaaagatttttgcagaatttctcaaaaatcagtttgtggattccagtagtatttatattgagagaaacaaattttagtccttctaactgtgctaaaacttggatatttgtgaaaactccttaccaccatacaagcatcagaagagctctcttgttgttagcacttattgtttgcaagaacagaatacatccttttatccttttatgaaaaatgacaagtgaaggcaaaaggggaaggttatttgatctggaagatgagtgttctgatgtggtggcttttgcaaaaatctttattggtgttgaaaactggaaaaaataactcatccagaattcatattgtcttgacaagaactatggttctctgtttttagatattgtggaaaatgtttttgggcatttttctctgattttatttcttctcccccacccctttttctaaaaaacaaacaaaaaaaaaaacacacaaaacaaaaacagaacaaaagaagagagaaggaaattttatcaattaaaaatgctgtgtgataaaatcccagcccagattgctcagctgtttgtacctgacttgccgcctgcataggagccagttctgttccttctgactagcccctcttcctccaggggagaacttccaaatgttaatttttttttttttgaaaatataaataattactattttgtactgtgtggtatctctggtcttttgtttcactcacctgccttgtctcttgggtctgagtcccttgcttaagggattttgaagtcctagttttcagcttgcagagattatgtctgaaatgcctaatgagtcgcagggatttgttgagactccgtaatctcaagttctctttgtgagctatcagcatctgccagtctcttgtcctccctgagtatctcacagtccatatcctgatgagggatcaggcccctacctctgccaaggcaagtaatggtagtgggcttttaaactgccccccgtatgttttaagacctaatccccacctcccttcttctaactaaatataaaaagatccaggggacataaatgtggagattaaataaagggaaattattgtctctaactggttctgtcattgacttgatgtgtttccagaaaagctaatactggagc

SEQ ID NO: 2 - protein, 374 aa, Homo sapiens
MSRVPSPPPPAEMSSGPVAESWCYTQIKVVKFSYMWTINNFSFCREEMGEVIKSSTFSSGANDKLKWCLRVNPKGLDEESKDYLSLYLLLVSCPKSEVRAKFKFSILNAKGEETKAMESQRAYRFVQGKDWGFKKFIRRDFLLDEANGLLPDDKLTLFCEVSVVQDSVNISGQNTMNMVKVPECRLADELGGLWENSRFTDCCLCVAGQEFQAHKAILAARSPVFSAMFEHEMEESKKNRVEINDVEPEVFKEMMCFIYTGKAPNLDKMADDLLAAADKYALERLKVMCEDALCSNLSVENAAEILILADLHSADQLKTQAVDFINYHASDVLETSGWKSMVVSHPHLVAEAYRSLASAQCPFLGPPRKRLKQS

SEQ ID NO: 3 - DNA, 5723 bp, Homo sapiens
ggcacggcgcaggggtggggccgcggcgcgcatgcgtcctagcagcgggacccgcggctcgggatggagggtgagtgagtaaacaagcctgggccccactgtctgttctcttttgaaaagctggacacctgttctgctgttgtgtcctgccattctcctgaagaacagaggcacactgtaaaacccaacacttccccttgcattctataaggtaggtaaagtatattagaatacattatttctcatctctgtctctcttagattacagcaagatggaaataccaaatccccctacctccaaatgtatcacttactggaaaagaaaagtgaaatctgaatacatgcgacttcgacaacttaaacggcttcaggcaaatatgggtgcaaaggtaaaaaataattcccaagtgaaagttaatcttttccatggctttgctaggctttgtatgtggcaaattttgcaaaggttcaagaaaaaacccagatcctcaatgaagaatggaagaagcttcgtgtccaacctgttcagtcaatgaagcctgtgagtggacacccttttctcaaaaaggtacttttgggagtttaagctcagtctccatgtcattgcttatatttcagtgtaccatagagagcattttcccgggatttgcaagccaacatatgttaatgaggtcactgaacacagttgcattggttcccatcatgtattcctggtcccctctccaacagaactttatggtatgtattgaaagcactgtggtggtaaatcacatctggtttggtttcaggtagaagatgagacggttttgtgcaatattccctacatgggagatgaagtgaaagaagaagatgagacttttattgaggagctgatcaataactatgatgggaaagtccatggtgaagaaggtagtggtactgaactccttggagacatctttcgaatcctcatgtttcagagatgatccctggatccgttctgattagtgatgctgtttttctggagttggtcgatgccctgaatcagtactcagatgaggaggaggaagggcacaatgacacctcagatggaaagcaggatgacagcaaagaagatctgccagtaacaagaaagagaaagcgacatgctattgaaggtacgtagcactggctcccttattcgagctttttatcatttccaattcaggcaacaaaaagagttccaagaaacagttcccaaatgacatgatcttcagtgcaattgcctcaatgttccctgagaatggtgtcccagatgacatgaaggagaggtagggaaagctgtcccacttaggcctcttatctctcttttcttaaataggtatcgagaactaacagagatgtcagaccccaatgcacttccccctcagtgcacacccaacatcgatggccccaatgccaagtctgtgcagcgggagcaatctctgcactccttccacacacttttttgccggcgctgctttaaatacgactgcttccttcaccgtgagtggggctactctgtttgaaacaacaagacagtttttgttttccagcttttcatgccacccctaatgtatataaacgcaagaataaagaaatcaagattgaaccagaaccatgtggcacagactgcttccttttgctggtatgttcttaagctgttctggcatcctgtctgtaatgtgtccactgcaggaaggagcaaaggagtatgccatgctccacaacccccgctccaagtgctctggtcgtcgccggagaaggcaccacatagtcagtgcttcctgctccaatgcctcagcctctgctgtggctgagactaaagaaggagacagtgacagggacacaggcaatgactgggcctccagttcttcaggtacgatgaacagaaaatgaagactattctgttatttcttcccttctcagaggctaactctcgctgtcagactcccacaaaacagaaggctagtccagccccacctcaactctgcgtagtggaagcaccctcggagcctgtggaatggactggggctgaagaatctctttttcgagtcttccatggcacctacttcaacaacttctgttcaatagccaggcttctggggaccaagacgtgcaagcaggtaccatctggaagcagcagaacaacaggcccgttggatttctcttccaggtctttcagtttgcagtcaaagaatcacttatcctgaagctgccaacagatgagctcatgaacccctcacagaagaagaaaagaaagcacaggcaagaagggttggacaaagtgtttgacctgtccttcctgtctttttcagattgtgggctgcacactgcaggaagattcagctgaagaaaggtaagttctaccagggcattcttcacatcctcctctgttttctttcctagataactcttccacacaagtgtacaactaccaaccctgcgaccacccagaccgcccctgtgacagcacctgcccctgcatcatgactcagaatttctgtgagaagttctgccagtgcaacccagactgtaagtgtgctacgttttctgtataagacttacctgtcttcccttcccaggtcagaatcgtttccctggctgtcgctgtaagacccagtgcaataccaagcaatgtccttgctatctggcagtgcgagaatgtgaccctgacctgtgtctcacctgtggggcctcagagcactgggactgcaaggtggtttcctgtaaaaactgcagcatccagcgtggacttaagaaggtgaggcctttcctcaggaccccactgagacttggccccctcttccccagcacctgctgctggccccctctgatgtggccggatggggcaccttcataaaggagtctgtgcagaagaacgaattcatttctgaatactgtggtgaggtgagtgctatagttttggcccacaatttgttatttgtctatctctctagctcatctctcaggatgaggctgatcgacgcggaaaggtctatgacaaatacatgtccagcttcctcttcaacctcaataatggtatgaagtcactttgtctcatcccgttttccttgatttactttatacagattttgtagtggatgctactcggaaaggaaacaaaattcgatttgcaaatcattcagtgaatcccaactgttatgccaaaggtgagtcccagtaacctgggaggtgagcctcgggtttatcctgcttgcagtggtcatggtgaatggagaccatcggattgggatctttgccaagagggcaattcaagctggcgaagagctcttctttgattacaggtgaggtgccagtaatggtccttgggttcctcttccctgctctgggacaggtacagccaagctgatgctctcaagtacgtggggatcgagagggagaccgacgtcctttagccctcccaggccccacggcagcacttatggtagcggcactgtcttggctttcgtgctcacaccactgctgctcgagtctcctgcactgtgtctcccacactgagaaaccccccaacccactccctctgtagtgaggcctctgccatgtccagagggcacaaaactgtctcaatgagaggggagacagaggcagctagggcttggtctcccaggacagagagttacagaaatgggagactgtttctctggcctcagaagaagcgagcacaggctggggtggatgacttatgcgtgatttcgtgtcggctccccaggctgtggcctcaggaatcaacttaggcagttcccaacaagcgctagcctgtaattgtagctttccacatcaagagtccttatgttattgggatgcaggcaaacctctgtggtcctaagacctggagaggacaggctaagtgaagtgtggtccctggagcctacaagtggtctgggttagaggcgagcctggcaggcagcacagactgaactcagaggtagacaggtcaccttactacctcctccctcgtggcagggctcaaactgaaagagtgtgggttctaagtacaggcattcaaggctgggggaaggaaagctacgccatccttccttagccagagagggagaaccagccagatgatagtagttaaactgctaagcttgggcccaggaggctttgagaaagccttctctgtgtactctggagatagatggagaagtgttttcagattcctgggaacagacaccagtgctccagctcctccaaagttctggcttagcagctgcaggcaagcattatgctgctattgaagaagcattaggggtatgcctggcaggtgtgagcatcctggctcgctggatttgtgggtgttttcaggccttccattccccatagaggcaaggcccaatggccagtgttgcttatcgcttcagggtaggtgggcacaggcttggactagagaggagaaagattggtgtaatctgctttcctgtctgtagtgcctgctgtttggaaagggtgagttagaatatgttccaaggttggtgaggggctaaattgcacgcgtttaggctggcaccccgtgtgcagggcacactggcagagggtatctgaagtgggagaagaagcaggtagaccacctgtcccaggctgtggtgccaccctctctggcattcatgcagagcaaagcactttaaccatttcttttaaaaggtctatagattggggtagagtttggcctaaggtctctagggtccctgcctaaatcccactcctgagggagggggaagaagagagggtgggagattctcctccagtcctgtctcatctcctgggagaggcagacgagtgagtttcacacagaagaatttcatgtgaatggggccagcaagagctgccctgtgtccatggtgggtgtgccgggctggctgggaacaaggagcagtatgttgagtagaaagggtgtgggcgggtatagattggcctgggagtgttacagtagggagcaggcttctcccttctttctgggactcagagccccgcttcttcccactccacttgttgtcccatgaaggaagaagtggggttcctcctgacccagctgcctcttacggtttggtatgggacatgcacacacactcacatgctctcactcaccacactggagggcacacacgtaccccgcacccagcaactcctgacagaaagctcctcccacccaaatgggccaggccccagcatgatcctgaaatctgcatccgccgtggtttgtattcattgtgcatatcagggataccctcaagctggactgtgggttccaaattactcatagaggagaaaaccagagaaagatgaagaggaggagttaggtctatttgaaatgccaggggctcgctgtgaggaataggtgaaaaaaaacttttcaccagcctttgagagactagactgaccccacccttccttcagtgagcagaatcactgtggtcagtctcctgtcccagcttcagttcatgaatactcctgttcctccagtttcccatcctttgtccctgctgtcccccacttttaaagatgggtctcaacccctccccaccacgtcatgatggatggggcaaggtggtggggactaggggagcctggtatacatgcggcttcattgccaataaatttcatgcactttaaagtcctgtggcttgtgacctcttaataaagtgttagaatccattttggcaagttgtgtactgtgtgctttggggctggaaggatccagggat

SEQ ID NO: 4 - protein, 747 aa, Homo sapiens
MEIPNPPTSKCITYWKRKVKSEYMRLRQLKRLQANMGAKALYVANFAKVQEKTQILNEEWKKLRVQPVQSMKPVSGHPFLKKCTIESIFPGFASQHMLMRSLNTVALVPIMYSWSPLQQNFMVEDETVLCNIPYMGDEVKEEDETFIEELINNYDGKVHGEEEMIPGSVLISDAVFLELVDALNQYSDEEEEGHNDTSDGKQDDSKEDLPVTRKRKRHAIEGNKKSSKKQFPNDMIFSAIASMFPENGVPDDMKERYRELTEMSDPNALPPQCTPNIDGPNAKSVQREQSLHSFHTLFCRRCFKYDCFLHPFHATPNVYKRKNKEIKIEPEPCGTDCFLLLEGAKEYAMLHNPRSKCSGRRRRRHHIVSASCSNASASAVAETKEGDSDRDTGNDWASSSSEANSRCQTPTKQKASPAPPQLCVVEAPSEPVEWTGAEESLFRVFHGTYFNNFCSIARLLGTKTCKQVFQFAVKESLILKLPTDELMNPSQKKKRKHRLWAAHCRKIQLKKDNSSTQVYNYQPCDHPDRPCDSTCPCIMTQNFCEKFCQCNPDCQNRFPGCRCKTQCNTKQCPCYLAVRECDPDLCLTCGASEHWDCKVVSCKNCSIQRGLKKHLLLAPSDVAGWGTFIKESVQKNEFISEYCGELISQDEADRRGKVYDKYMSSFLFNLNNDFVVDATRKGNKIRFANHSVNPNCYAKVVMVNGDHRIGIFAKRAIQAGEELFFDYRYSQADALKYVGIERETDVL

SEQ ID NO: 5 - DNA, 10151 bp, Homo sapiens
agcgcttcccgtgtgcggggcttcccacaatgcaccgggccggcagtggcggcgaccgcggcggcgctctagctgcggcatgtctgcgtctctactgctctgggaggaggaagagaaggaggaagaggaggaggaggaggagggggaggaagaggagaaaggcgcaggggtgggagctgttgccgaagctgccacagcaaaagttctcccccctccccccttcccctcctctcaaggcccctagaaaggttggagctgccgccgcctgcagtcggtgaccgcgcgactcggcgcccgcccgcggtaaagcgctcggcctggcaggcccaacctgtatttgtgtacttttgtaggatagagggaggaatcagcagcttggaaattcaagcacgtgatctggcgggatgggcgtttgcctaacgtatttaatggaggtaattcagcattctttgagtcaatttgttgtgtgtgtgttttattgtaggaatcggatggcataagtgattaaggtggtattgaggatttctgaagcctatgaaaggtagaaactcaaccatgatttctttttcaactctacagcattcctttccttgaagtcttcgtttttaccttagtctcgggtaattttaatacttatttttcatatttgttgccaaacttggttttctaggcagttatacttaagcatgaacattgacgacaaactggaaggattgtttcttaaatgtggcggcatagacgaaatgcagtcttccaggacaatggttgtaatgggtggagtgtctggccagtctactgtgtctggagagctacaggattcagtacttcaagatcgaagtatgcctcaccaggagatccttgctgcagatgaagtgttacaagaaagtgaaatgagacaacaggatatgatatcacatgatgaactcatggtccatgaggagacagtgaaaaatgatgaagagcagatggaaacacatgaaagacttcctcaaggactacagtatgcacttaatgtccctgtaagtaattcctttataagatattaattggcttttctttttttaaacagataagcgtaaagcaggaaattacttttactgatgtatctgagcaactgatgagagacaaaaaacaaatcagagagccagtagacttacagaaaaagaagaagcggaaacaacgttctcccgcaaaagtaagacaatgtattaaggtggtaattttgcctttttttttttgaactagatccttacaataaatgaggatggatcacttggtttgaaaacccctaaatctcacgtttgtgagcactgcaatgctgcctttagaacgaactatcacttacagagacatgtcttcattcatacaggtatttcttgaattaaaatgggctttttgtggatgtgaaattattttaaggtgaaaaaccatttcaatgtagtcaatgtgacatgcgtttcatacagaagtacctgcttcagagacatgagaagattcatactggtgagtgttaaccacccttactaggttaaactttctatattatgtgctaggtgaaaaaccatttcgctgtgatgaatgtggtatgagattcatacaaaaatatcatatggaaaggcataagagaactcatagtggagaaaaaccttaccagtgtgaatactgtttacaggtaagagagatggcttgaaattcttcactaccatcctctgaattcaacagtatttttccagaacagatcgtgtattgaaacataaacgtatgtgccatgaaaatcatgacaaaaaactaaatagatgtgccatcaaaggtggccttctgacatctgaggaagattctggcttttctacatcaccaaaagacaactcactgccaaaaaagaaaaggcagaaaacggagaaaaaatcatctggaatggacaaagagagtgctttggacaaatctgacctgaaaaaagacaaaaatgattacttgcctctttattcttcaagtactaaagtaaaagatgagtatatggttgcagaatatgctgttgaaatgccacattcgtcagttgggggctcgcatttagaagatgcgtcaggagaaatacacccacctaagttagttctcaaaaaaattaatagtaagagaagtctgaaacagccactggagcaaaatcaaacaatttcacctttatccacatatgaagagagcaaagtttcaaagtatgcttttgaacttgtggataaacaggctttactggactcagaaggcaatgctgacattgatcaggttgataatttgcaggaggggcccagtaaacctgtgcatagtagtactaattatgatgatgccatgcagtttttgaagaagaagcggtatcttcaagcagcaagtaacaacagcagggaatatgcgctgaatgtgggtaccatagcttctcagccttctgtaacacaagcagctgtggcaagtgtcattgatgaaagtaccacggcatccatattagagtcacaggcactgaatgtggagattaagagtaatcatgacaaaaatgttattccagatgaggtactgcagactctgttggatcattattcccacaaagctaatggacagcatgagatatccttcagtgttgcagatactgaagtgacttctagcatatcaataaattcttcagaagtaccagaggtcaccccgtcagagaatgttggatcaagctcccaagcatcctcatcagataaagccaacatgttgcaggaatactccaagtttctgcagcaggctttggacagaactagccaaaatgatgcctatttgaatagcccgagccttaactttgtgactgataaccagaccctcccaaatcagccagcattctcttccatagacaagcaggtctatgccaccatgcccatcaatagctttcgatcaggaatgaattctccactaagaacaactccagataagtcccactttggactaatagttggtgattcacagcactcatttcccttttcaggtgatgagacaaaccatgcttctgccacatcaacacaggactttctggatcaagtgacttctcagaagaaagctgaggcccagcctgtccaccaagcttaccaaatgagctcctttgaacagcccttccgtgctccctatcatggatcaagagctggaatagctactcaatttagcactgccaatggacaggtgaaccttcggggaccagggacaagtgctgaattttcagaatttcccttggtgaatgtaaatgataatagagctgggatgacatcttcacctgatgccacaactggccagacttttggctaaaaaaaaaaaaaaagtgtaaataatactggcactttagaacagattaatcaagagtggggttactctgtgtaaatggagtgctgtacagatttaagagcaatgcgtaataacaagttaagctgatatgaatagcaagataatccaataactgcatttcgtttggttagtcagcattctttgaactgccttacatgttgtcacctttatagaagcaatgcattacttgttttagatcagaaacttgctattccacccacaccaagttaaaaaggaaaaaaaaaagactttcgcacaattgtttcctaactgataacattgtacattcttaggagattagtaattgtgtgaaatttactcatactgtttctaagtttttcagcatagtcattgcacttcagcagggaatctgagtatactttacagacagagtgaacttaaaagtttaatgtcaagagattatggcttaaataaattagtgtgtcctatagggggaaaaaaaccaagaaaccaccttttaaaaagaatgatatgccatatacccttgattttcattttgcattatattgacgtgtttttttgaaggaaaaaaagtaataaaaatctgatagtctaagactccactatttaaaagcctaattactttaaaaatatgcatactttcagaacttttaccaaaacacacaactgttgaagcagtcacttctctatggaagtatgcatattggtgtcagtttctttgtacagttgtacttagatattttttatgatttttcatgtgcaggtatcaaggttttgaagttttagtaaaagaaattctgtagattacattcccaagaacataatgcttacacaaaatgtatattccacgttttaaagcttaattgtattttactttacatatacacttcagttaacatagagcacttagaatctatttggtatttttgatttctcaaagtaaaaaaaaaattagatttttaggtttgatatggttgtgtcataatcatctcgtaattgacaattttaacttttggcaataaaaggaaattgggatatctttggaactgtaaaacctggtttaatctttttctttctagactcttgatagattggataattaaacagtatccagagaaactaaagaaatgggcattttaattgcatattttatcttgagttactttttaatgaacactgctcattacaactttacaaaccaaaagtgtttactaattccagtaactaccatttctttttcagctagatgagtgatcggaaaatttttgttgcatttcaaaatctaaataaactacagactttatccttataccatgaatgtttttttttaatactctgtcttaaaataatacagcatggtacaataaatagtgcttttatgtatatatacacacacacacttttctgaagaatgatggtttgagttatgcgttgggcagttttgatttttggaagttatattagttattgactctttgttacaactttttcatttttacattttaaattttctgccatcgttgtcacaatgcacatcctgatatagcaccagtgaaaatctaaaattaatacccttggaaaatgaaaatattcttaatttccattttgactcttatactggccctatagctctgcagtctttgtttcaatatagaatatgttatccatttaaattattttttcttttatttaatgttatcccaagactgttctcataaagaaagggaagaaataactatccacccttaacatccttcatttattttgaatatattggtgtttttatgataaggaatataaattatatttaatgtggtttcctgtatactttggttattaagttttgcttgaaatagtagttttcccgtttgacagctttctttgctgccaaagttttcaagaatttaagacttctttgaagtaaatatttaagttctgccattattgactcttaagattgtgtgtgttgtgttgtgcattacataattacaaaaaggcttcattcaggtgatacgttaaaaatggaactgtgctcaccctaaataggcattggtatttttctcttttggtgagagtaggcatttatttcttgagttgttttggagcctgatcaaaaattttgttcatggagaacttgatgcaattttgatacagtggagaggtttttttcggttgttttaacatcaccagcatagtttttagaatgtgactcttgctgagcatttagggtgagcttgggaaggaaggcctttgaaaatggtagttatgcaagcagactttcaggtgttgcattcctgttttcaacacatttctttaaatctacataatggcagacttttctaccaggttataagcagtttttagataagtgatactcagccagcataacttattgacttaccatttacgtatagtcatcactctcttactgtgaaattccaaatgcctaccagagttaccttgttctatcataatatgacaaacatctcaacagttttggatttccccactttggttcagaaggttatttaatttctatctgggcattaatggagaaaataagtagccttgtgtgctgcttcagattgaaacatggaggatatgagatattcttctgcaattcatgtttctcatttctcaaagtgagcacattgttctataaaaacatgctggtacacccttaaacttttgatcaatctgagtgaggtgtgcttttcctcttgggacctactcacgtttgaagattggaaacatcacgttaggcgaggcagtatctcttgaacatcttctaaagggttttttaaaaccttattctcacatatttcatttgtcttgaatatttaagtggctcttaaacgttttgggtctacatgcaaatgtggtacttaacaaggtaggaatccatcttcttagctctggctgagggtgccagccatcggtcaggtcatttttatctcaagaggcagaaggcagttatgtcaagaattgtgctcagggcaggatttcgtttccacagaggagagacacttgcagagtgccagctaggttagatcttttgccgcttctttcatttgattaatttgggtttttaaagggctgtttaaaaaaaaaaaaaaaaggccgggaaactttaaaagtaggcattactgtagtactgcatttcttagaacattttaaactagaactcatttttttttcacagtatatttacttgaagaagcactataataatcattgagaagtattttgagtctgaaatttaatttaattttccgtttcaaattgctttatttcagggaaataattttccagttgttttgctatattctgcaaataaaaaccgtgtttccttttttcacttaaactttggtaggaaacaaactaaagcagacaaacatttcttgttatgtttgttgctttctttaatccaatggataaaaaagtaaaaccctgtaaacattattttatttttttatgcaataccatgctgtaaatatggttcatcaaataaggatgtacctatgattgaatctttaattctgcacagttagagtttatatataaacgtgtcttgacaatcaaggacttttatgtgagtcttcctttatgatgtttattaatgttatgcattccatttgttttgaagtgagtaccaatgtgctaatttgtattgtctgaaagtatgtaatgtttttacagcttgtttttaagaatctgtaaatatgtacaaacaaatcacagtactgctttatgttagaaggcatatgatgttgtactgtatgtaagcaataatacatagcagtgctaactttacaagtagcatcaggaaagttctaaaaacatttcagagttattaattatccttatttcatttaataatgattatctttaatcagtttttataagcaaattccattgttcctcctattggaacgtaacactgtttacacagcataattgaagttgcaggggagacaggctaaatctgacttcatctgtactcactttcactaagcattgaatggccttaccactcttctgcaagatggaggaagaccgcaaaacttagtgcccatcacactgaaggaagggatgtggttttttttcctgcttctgtactgctacctaactttgtgatttgctttggattttaatatttgtttctgttttagattcaagcccgtaagttttgaggtgacttcagctccattgtgaaatagatagttctcttcatatttcataactacatttcaataattctaaactttctacattgatttttagatacttatttttcaagcttggtttctcagctgatcagatgaatttattcaacttcaatacaaagaaagacaaacctattatagtttcaagtgagtagatattatactgtacaggacagctatttgaacacacacatcactgctttagaaataaaaccgccaatttataaatgtatatgacctacattttataggaaaaaatgtttttaaatgctagtcatttatataatgtgctttgaaggatttgctagtccacttctgtcactttttagtacactggtatcttttatatgtaatgtatgctttttattattgtagcaaagcatttcagtagaaagaattttgcaacaatatgggggaaatttttcattgttggatttgaattatactggattttatctgtgtagtcttacttgtctttattttaatgctgtctgaagggaacttggtatccaaataaagcaggtaacctcattttacttcaagggcatactgtgtagtgctgaatttaatctgaaaatctgatgattttgaaaagaaaaacaagtttataaacattgtacagaagaaattaaatgtgtgtgatggaaatcctgatggtggagcacgttgaattttctgatatatagtgttattttcctgggatcccctatgccttctttgtatttcaactaataaaggaaaaaccctgtcattaaaactggtaagataataggatattctgattatacagtattactattcctatcccattttctgaccttttctcaatcactgtaaatttttattttctatttcctattgataattaaatatgtacataatatagacactgttgtatagaactaaatgaattgggttgctatgcttgagtgggtagtgaaatggaaatatttgatgaatactgagagttcctaaaatgctagagcaaatttccgagggaaaggggggcatcgtgtgtcttgggaaggttgcagcaggttgtttcttcaactccctaagaaatcattgggaaggactcaactgcaaactcgggatcagatacaaaatgggtctttccttcccctctcccccaaaaaataggagcactaaattttaaaatctactcaggtgacagttgctttgcaatgaaacttatcacattgaaattttcagtgttaaataaaaagagatttgtgatatttttaaatataaacttttacatcagtagtcagcagcctgacaattgaaatttggtaagtcgatatattttaaaatattttgctctccaaatagaattgtttttaaacaattgggaggttttttgtttctttaaatatgttttaattgtcattgtaaaaacaaaatcttgcttgaccctatattatgccatgaatgaattgctgagcctttataatacagtgacagcttgtttcatgcatagtcatggaacatagagatgttctttaaactgacctattgataagaggtttaatgagtttcacggctttcaacactattgtcatatagttatctttgccatttggagtttataaaaccattttatgtattgtgatactaaaggaagttgttttgctttactttaaattgaattattagactaatatttgcaaattctgcattttaaatgtggacactggctgtttgaaaaataaaatatagaatacagttttatggataattttcgagctgaatttttcacaatattctgaaccaaataacgaatggtaaatatgcaaaaatcatggtgcatagatataaccgtaaagagaaaagaatttctgtgtggaattaacagttacacaaattgggtaacaattatcagggcctttattatagctttatgtggaatgttattctaaaatgcaagagtcaaatgttactgtcattgaatattttagacttgaaacgtgtttatcatagagcgaagaaaatatgtgttcttttctttacagataagactctgttaacccactgtcagcatacgtgggatttcttttcttttttctttcattagtggagatttgttttcatccatctaccaccttgccagtaccctagcttgtgaccagcgggatgcattgtaagaaagttgtctgtggttaggagtgcagtctgggtccatggaacaaatttaaactaattggccctcgcaatgatcagtcctaggaccctggccttattattagcatggtgctttaaccaattgtacataataataccactgatagtctactaatgcatttcctagatcccagtttttcttgaatgattaggaatggtggggagaggggaggggagatattctacatgatacttctccaatcttctaaagattataagaaaaaataaaaaattgaagtcacttgaattaatgtgttgtcattgagtcttactcgacaatttatcatgcacaaagtgattatgaagattttcctgattatatgtttggattgaatttaaaaattttttttttcagaatgctggctgttctgttttatttcttctctggtgaacctgataggatat

SEQ ID NO: 6 - protein, 794 aa, Homo sapiens
MNIDDKLEGLFLKCGGIDEMQSSRTMVVMGGVSGQSTVSGELQDSVLQDRSMPHQEILAADEVLQESEMRQQDMISHDELMVHEETVKNDEEQMETHERLPQGLQYALNVPISVKQEITFTDVSEQLMRDKKQIREPVDLQKKKKRKQRSPAKILTINEDGSLGLKTPKSHVCEHCNAAFRTNYHLQRHVFIHTGEKPFQCSQCDMRFIQKYLLQRHEKIHTGEKPFRCDECGMRFIQKYHMERHKRTHSGEKPYQCEYCLQYFSRTDRVLKHKRMCHENHDKKLNRCAIKGGLLTSEEDSGFSTSPKDNSLPKKKRQKTEKKSSGMDKESALDKSDLKKDKNDYLPLYSSSTKVKDEYMVAEYAVEMPHSSVGGSHLEDASGEIHPPKLVLKKINSKRSLKQPLEQNQTISPLSTYEESKVSKYAFELVDKQALLDSEGNADIDQVDNLQEGPSKPVHSSTNYDDAMQFLKKKRYLQAASNNSREYALNVGTIASQPSVTQAAVASVIDESTTASILESQALNVEIKSNHDKNVIPDEVLQTLLDHYSHKANGQHEISFSVADTEVTSSISINSSEVPEVTPSENVGSSSQASSSDKANMLQEYSKFLQQALDRTSQNDAYLNSPSLNFVTDNQTLPNQPAFSSIDKQVYATMPINSFRSGMNSPLRTTPDKSHFGLIVGDSQHSFPFSGDETNHASATSTQDFLDQVTSQKKAEAQPVHQAYQMSSFEQPFRAPYHGSRAGIATQFSTANGQVNLRGPGTSAEFSEFPLVNVNDNRAGMTSSPDATTGQTFG

SEQ ID NO: 7 - DNA, 18 bp, Artificial Sequence
  synthesized
ccagatcaaagccacaac

SEQ ID NO: 8 - DNA, 20 bp, Artificial Sequence
  synthesized
ctggacgatagagtaagacc

SEQ ID NO: 9 - DNA, 20 bp, Artificial Sequence
  synthesized
acacctgcttttttgactcg

SEQ ID NO: 10 - DNA, 20 bp, Artificial Sequence
  synthesized
aaccagtggaaagagaatgc

SEQ ID NO: 11 - DNA, 20 bp, Artificial Sequence
  synthesized
tcttggttgaccaaaaccac

SEQ ID NO: 12 - DNA, 20 bp, Artificial Sequence
  synthesized
ggcccctcctgcaaattatc

SEQ ID NO: 13 - DNA, 20 bp, Artificial Sequence
  synthesized
tttgggagggtctggttatc

SEQ ID NO: 14 - DNA, 21 bp, Artificial Sequence
  synthesized
ccacatatgaagagagcaaag

SEQ ID NO: 15 - DNA, 20 bp, Artificial Sequence
  synthesized
caggctttggacagaactag

SEQ ID NO: 16 - DNA, 20 bp, Artificial Sequence
  synthesized
tacacagagtaaccccactc